(19) United States
(12) Patent Application Publication
(10) Pub. No.: US 2007/0155774 A1
(43) Pub. Date: Jul. 5, 2007

(54) Oral formulations of desoxypeganine and thereof uses

(76) Inventors: Joachim Moormann, Warne (DE); Klaus Opitz, Munster (DE); Hans-Rainer Hoffmann, Neuwied (DE)

Correspondence Address: Sean F Mellino;D Peter Hochberg, 1940 East 6th St-6th Floor, Cleveland, OH, 44114, US

(21) Appl. No.: 10/580,485

(22) PCT Filed: Nov. 8, 2004
(86) PCT No.: PCT/EP04/12606

§ 371 Date: May 24, 2006

(30) Foreign Application Priority Data
Nov. 24, 2003 (DE) 103 54 894.7

(51) Int. Cl. A61K 31/4745

(52) U.S. Cl. 514/292

(57) ABSTRACT

Orally administrable film-shaped medicaments which contain the active substance deoxypeganine or/and a deoxypeganine derivative. The medicaments can be used for transmucosal administration of active substances.

### CROSS-REFERENCE TO RELATED APPLICATIONS

This application is a National Stage application of International Application No. PCT/EP2004/012606, filed on Nov. 8, 2004, which claims priority of German application number 103 54 894.7, filed on Nov. 24, 2003.

### BACKGROUND OF THE INVENTION

1. Field of the Invention

The invention relates to oral film-shaped medicament formulations for administration of deoxypeganine or of its salts and derivatives, and to the use of said medicaments for treating diseases or symptoms.

2. Description of the Prior Art

Deoxypeganine (1,2,3,9-tetrahydropyrrolo[2,1 -b]quinazoline; empirical formula C₁₁H₁₂N₂) occurs in plants of the Zygophyllaceae family. On the basis of its pharmacological properties, deoxypeganine is included in the group of reversibly acting cholinesterase inhibitors. It also acts as mono-amino oxidase inhibitor. Deoxypeganine (also called deoxyvasicine) is being taken into consideration as a medicament for therapeutic purposes, e.g. for treating drug addiction and drug dependency (DE-A 199 06 978), for the therapy of nicotine dependence (DE-A 199 06 979) and dependence on alcohol (DE-A 199 06 974), for treating psychiatric or cerebral pathological manifestations (DE-A 101 19 863), for the therapy of Alzheimer's dementia (DE-A 199 06 975), clinical depression (DE-A 101 63 667) or schizophrenia (EP-B 0 584 285), as well as for the prophylaxis of poisoning by organophosphorous cholinesterase inhibitors (DE-A 199 24 951), or for treating chronic fatigue syndrome (U.S. Pat. No. 5,312,817).

Deoxypeganine is preferably obtained by isolation from Syrian rue (Peganum harmala) or by chemical synthesis (e.g. SARGAZAKOV et al.; Khim. Prir. Soedin. 4 (1990), 506-507; MORRIS et al.; J. Amer. Chem. Soc. 57 (1935), 951-954). Deoxypeganine is known to the pharmaceutical art from the literature and, in particular, from patent specifications.

Using conventional administration forms such as tablets, capsules, suspensions or solutions for the purpose of oral administration of deoxypeganine is disadvantageous insofar as deoxypeganine is absorbed mainly from the intestine, thus being subject to “first pass” metabolism. In addition, the use of the aforementioned administration forms is not possible, or only conditionally possible, in those cases where a person experiences pain on swallowing or where a person refuses to swallow such medicaments.

It has therefore been proposed to administer deoxypeganine by a transdermal therapeutic system (TTS) (DE-C 199 06 977). The disadvantage here is that therapeutically effective plasma levels are built up only after a considerable delay in time. However, in many cases it is essential that the onset of action occurs as quickly as possible.

### SUMMARY OF THE INVENTION

The object of the present invention is therefore to provide administration forms for administering deoxypeganine (or a salt or derivative thereof) which are suitable for treating the diseases and symptoms set out at the start, while avoiding the above-mentioned disadvantages of known administration forms, especially tablets, as far as possible.

It has surprisingly turned out that these objects are achieved by film-shaped medicaments and by using such medicaments for treating diseases and symptoms discussed in the present application.

The oral film-shaped medicaments (also called “wafers”) surprisingly enable transmucosal absorption of deoxypeganine (and its salts or derivatives) in the region of the oral mucosa. The film-shaped medicaments are preferably applied buccally or sublingually. The inventive preparations largely avoid the first-pass metabolism and enable a rapid onset of action (within approximately 5 seconds to 10 minutes). The medicaments of the invention are applied in the oral cavity, whereupon the active substance(s) is/are released from the film-shaped preparation as a result of the action of saliva, and subsequently absorbed via the oral mucosa. The invention also encompasses mucoadhesive film-shaped preparations which are applied to the oral mucosa and at least temporarily remain adhering thereto. In this case, the active substance delivery can, in addition, take place directly via the mucosal region of the application site, where the film-shaped preparation is in direct contact with the oral mucosa.

Although oral, especially buccal or sublingual, administration is preferred, the invention also encompasses administration forms which are intended for application to other mucosal surfaces (e.g. rectal, vaginal or intranasal) of the human or animal body and which enable the transmucosal administration of deoxypeganine.

It is advantageous that the medicaments of the invention can be administered in a simple, inconspicuous and safe manner, since unlike with tablets it is not necessary to use additional liquid for intake. In particular, film-shaped preparations of small thickness (e.g. less than 0.1 mm) are felt to be pleasant by the persons being treated.

### DETAILED DESCRIPTION OF THE INVENTION

The medicaments of the invention preferably contain the active substance deoxypeganine in the form of one of its water-soluble, pharmaceutically acceptable salts; deoxypeganine hydrochloride and deoxypeganine hydrobromide are particularly preferred. Deoxypeganine may, however, also be contained in the medicaments in the form of its free base. The invention further provides for the use of deoxypeganine derivatives, possibly in the form of pharmaceutically acceptable salts.

Deoxypeganine and its salts can be produced or isolated in accordance with one of the initially mentioned methods or it can be purchased on the market.

Suitable derivatives of deoxypeganine are, for example:
7-bromodeoxy-peganine (Synthetic Communs. 25(4), 569-572 (1995));
7-halo-6-hydroxy-5-methoxydeoxypeganine (Drug Des. Disc. 14, 1-14 (1996); Halo=Br, Cl, F or J), and the derivatives of deoxypeganine described in Ind. J. Chem. 24B, 789-790 (1985).

The medicaments according to the present invention may optionally contain a combination of two or more of the aforementioned active substances or active substance salts.

According to a further embodiment it is provided that the medicaments of the invention additionally contain at least one further active substance, in coordination with the given indication. Particularly suitable for this purpose are active substances from the group of the acetylcholinesterase inhibitors, which comprises galanthamine, pyridostigmine, physostigmine, neostigmine as well as the pharmaceutically acceptable salts of the aforementioned active substances.

Furthermore, the inventive medicaments may additionally contain at least one active substance that is not selected from the group of the acetylcholinesterase inhibitors; thus, for example, film-shaped preparations used for treating nicotine abuse may additionally contain opiate antagonists.

The overall active substance content of a film-shaped preparation according to the invention preferably amounts to 0.5 to 40%-wt, more preferably 5 to 30%-wt. The active substance dose contained in a single preparation is preferably in the range of 1 to 500 mg, particularly 10 to 300 mg.

The film-shaped medicaments preferably comprise at least one polymer-containing layer which serves as an active substance reservoir and which contains the active substance(s) and is able to release it/them upon the action of saliva. The polymer portion of this polymer-containing layer amounts to 10 to 90%, preferably 20 to 70%-wt. and particularly preferably 20 to 60%-wt.

In the simplest case the inventive preparation only consists of a single, active substance-containing layer. However, the invention also encompasses embodiments with a two-, three- or multilayer structure of which at least one layer contains active substance. The various layers may differ from one another in terms of their active substance content (type, concentration), their mucoadhesive properties, disintegration properties, solubility, etc.

“Film-shaped” means that the inventive medicaments, unlike conventional tablets, are of small thickness and are preferably bendable. Furthermore, after having absorbed moisture they are generally capable of conforming to the irregular surface contour of the oral mucosa. The total thickness of the active substance-containing films (in the condition prior to application) is preferably 0.05 to 3 mm, especially preferably 0.1 to 1 mm, and especially 0.1 to 0.5 mm. The shape of the surface of the individual medicaments may be round, oval, triangular or quadrangular, or polygonal. The extension of their surface area is preferably in the range from 0.5 to 20 cm², especially in the range from 1 to 10 cm².

Polymers suitable for producing the above-mentioned polymer matrix may be selected, in particular, from the following group: polyvinyl alcohols; polyvinyl pyrrolidones; polyvinyl acetate; polyethylene glycols; polyethylene oxide polymers; polyurethane; polyacrylic acid, polyacrylates, polymethacrylates; poly(methyl vinyl ether-maleic anhydride); cellulose ether, particularly ethyl cellulose, hydroxyethyl cellulose, propyl cellulose, carboxymethyl cellulose, Na-carboxymethyl cellulose, hydroxypropyl cellulose, hydroxypropyl methyl cellulose, hydroxypropyl ethyl cellulose; cellulose acetate; polysaccharides such as starch and starch derivatives; natural gums; alginates, pectins, gelatine. The aforementioned components may be used alone or in combination.

The inventive medicaments may additionally contain one or more auxiliary substances which are known to those skilled in the art and which may be selected, in particular, from the following groups: emulsifiers (e.g. polyethoxylated sorbitan fatty acid esters, polyethoxylated fatty alcohols, lecithin); plasticizers (e.g. polyethylene glycol, glycerol and other polyalcohols, higher alcohols such as dodecanol, undecanol, octanol; sorbitol, mannitol and other sugar alcohols, dexpanthenol; triglycerides), fillers (e.g. highly dispersed silicon dioxide, titanium dioxide, zinc oxide, chalk, starch); colourants; sweeteners and flavourings; wetting agents; preservatives, pH-regulating agents and antioxidants; disintegrants; substances improving absorption via the mucosa (e.g. fatty acids and fatty acid esters; polyhydric alcohols such as propanediol; tocopherols; ethereal oils such as menthol).

The weight percentage of these auxiliary substances may amount to up to 60%-wt, especially 5 to 40%-wt, in each case relative to the entire preparation. By adding the above-mentioned auxiliary substances, whose action is known to the skilled artisan, it is possible to influence the chemical or physical properties of the active substance-containing films such as capability of swelling, diffusion properties, mucoadhesive properties, flexibility and ability to disintegrate.

According to a preferred embodiment, the film-shaped medicaments are mucoadhesive or have at least one mucoadhesive outer surface, which enables these medicaments to adhere firmly to the oral mucosa. The mucoadhesive properties are essentially determined by the type of the polymer(s) forming the mucoadhesive layer as well as by the relative portions of these polymers In addition, these properties may be modified by the above-mentioned auxiliary substances (e.g. fillers, plasticizers). Preferably, the mucoadhesive layer also contains active substance.

It may be advantageous to combine a mucoadhesive layer with a non-mucoadhesive layer. By providing a non-mucoadhesive outer surface it is possible to prevent unwanted adherence to neighbouring mucosal areas (e.g. tongue).

Suitable polymers for producing a mucoadhesive layer may be selected from the groups listed in the following: polyvinyl alcohols; gelatine; polyvinyl pyrrolidones; polyacrylamide; polyacrylates; natural rubbers; starch and starch derivatives, pullulan; cellulose derivatives such as hydroxypropyl methyl cellulose, hydroxypropyl cellulose, sodium carboxymethyl cellulose), methyl cellulose, hydroxyethyl cellulose and hydroxypropyl ethyl cellulose; as well as combinations of the aforementioned polymers.

The mucoadhesive properties may furthermore be modified by suitable auxiliary substances known to those skilled in the art.

According to a further embodiment of the invention it is provided that the film-shaped medicament is soluble in aqueous media, especially in saliva. In this way it is possible to achieve a quick release of active substance. The preferred embodiment here is one where the dissolution takes place within 1 s up to 5 min, especially preferred within 3 to 30 s.

As an alternative, the medicament may be formulated as a rapidly disintegrating administration form which quickly disintegrates in aqueous media, especially in saliva, preferably within 1 s up to 5 min, especially preferably within 3 to 30 s. The solubility or disintegratability relates to the conditions present in the oral cavity with respect to temperature (approx. 35 up to 40° C.).

According to a preferred embodiment, the film-shaped medicaments are characterized by the fact that following application they release the active substance(s) contained therein into the oral cavity within 30 min, preferably within 15 min, especially preferably within 5 min, in such an amount that an effective plasma level is achieved.

If the film-shaped preparations are to enable a longer-lasting active substance release, they are advantageously formulated as mucoadhesive, slowly soluble or slowly disintegrating films which dissolve or disintegrate only after a number of hours (e.g. after 1 h, 6 h or 12 to 24 h). The invention also encompasses film-shaped medicaments which are insoluble or non-disintegratable under the above-mentioned conditions. In this case, the active substance release takes place exclusively by diff-usion of the active substance from the film into the environment. The release of active substance takes place with a delay in time, preferably over a period of up to 8 h, especially up to 24 h. Depot action may optionally also be achieved by encapsulating the active substance in particles (e.g. polymer particles), whose envelope slows down the diffusion.

Furthermore, it is provided according to a particularly preferred embodiment that a film-shaped medicament has at least one rapidly disintegrating or freely soluble layer as well as at least one slowly or non-disintegrating (or essentially insoluble), preferably mucoadhesive, layer, with the two layers containing active substance. In this way it is possible to combine a rapid initial dose with a maintenance dose of the active substance.

The above-mentioned soluble or disintegratable medicaments, too, may be provided with mucoadhesive properties, as has been mentioned. In this way it is achieved that such a preparation firmly adheres to the site of its application in the oral cavity until it has dissolved or disintegrated.

The solubility and disintegratability are essentially determined by the type of the polymer(s) forming the respective layer(s), as well as by the relative portions of these polymers; additionally these properties may be modified by the above-mentioned auxiliary substances (e.g. fillers, plasticizers). It is preferred that the soluble or disintegratable layer also contains active substance.

According to a further embodiment, the film-shaped medicaments are capable of gelatinizing or swelling in aqueous media, particularly in saliva. It is thereby possible to achieve a retardation of the active substance release.

To produce water-soluble (or disintegratable) film-shaped preparations or layers of such preparations, polymers from the following group are especially suitable: polyvinyl alcohols, polyvinyl pyrrolidones, polyethylene oxide polymers, polyacrylamides, polyethylene glycol, polyvinyl acetate, polyacrylic acid, polyacrylate; starch and starch derivatives, dextran; cellulose derivates (see above; especially ethyl cellulose, propyl cellulose, carboxymethyl cellulose); gelatine, and other gel-forming proteins; natural gums, pectins, alginates, pullulan, carrageenan, xanthan, tragacanth, chitosan, agar-agar, agarose. The aforementioned substances may be used alone or in various combinations, including combinations with auxiliary substances. They can further be used for producing the above-mentioned gelatinizable or swellable films or layers, optionally also utilizing auxiliary substances.

According to a further embodiment it is provided that the inventive film-shaped preparations are present as solidified foams. The production of such foams is described in DE-A-100 32 456, for example.

The inventive film-shaped medicaments may be obtained, for example, by applying the following method:
Preparing a liquid coating mass (solution, dispersion) containing polymer(s), active substance(s) and possibly auxiliary substances; by stirring and, if required, heating;
coating this mass onto an inert support (e.g. using doctor knife, roller application, spraying or extrusion methods) so that a thin film layer is obtained;
drying;
separating dosage units of the desired surface area and active substance content (e.g. by cutting or punching).

For example, to obtain a film which is composed of two or more layers, initially a first layer is prepared as described above and dried. The coating mass for the second layer is then applied to the dried layer and dried.

The inventive film-shaped medicaments may be used advantageously for treating diseases or symptoms caused by acetylcholine deficiency or where such deficiency occurs. They are further suitable for the treatment of diseases where a deficiency of endogenous amines occurs and/or which can be favourably influenced by inhibition of monoaminoxidase

The film-shaped medicaments are particularly suitable for treating the diseases and symptoms mentioned at the start, as well as for the above-mentioned prophylactic measures.

The inventive film-shaped preparations may be used, in particular, for the pharmaceutical therapy of the following diseases and symptoms:

Alzheimer's disease (especially Alzheimer's dementia); depression; chronic fatigue syndrome, disturbed sleep, schizophrenia; mania; Parkinson's disease; disorders of the central nervous system, particularly impaired memory, caused by the action of psychotropic substances, particularly intoxications with such substances; poisonings by neurotoxins or warfare agents (especially organophosphorous substances); alcoholism or nicotine dependence, abuse of other chemical substances; treatment for reduction of the craving for alcohol or for the reduction of the craving for nicotine.

To treat persons (or animals) suffering from one of the above-mentioned diseases or showing one of the above-mentioned symptoms or who for other reasons require treatment with a cholinergic active substance acting on the central nervous system, the person (or animal) to be treated is orally administered a therapeutically active dose of the active substance deoxypeganine (and/or one of the above-mentioned salts or derivatives) in the form of a film-shaped medicament, as described above.

To this end, the film-shaped preparation is introduced into the oral cavity (buccally, sublingually) and, in the case of mucoadhesive films, adhered to the buccal mucosa. Other regions of the oral mucosa (e.g. palate, sublingual, gingival) are also suitable as application sites. Application is repeated as often as required, e.g. in intervals of, preferably, 1 to 6 h. The daily dose of deoxypeganine, possibly in the form of a pharmaceutically acceptable salt (and/or deoxypeganine derivative(s)) is generally in the range from 50 to 750 mg.

A film-shaped preparation according to the invention may, for example, be obtained with the following formula. The components are dissolved in water under heating and the resultant solution is coated onto a smooth, inert support (polished steel tape). After drying, (approx. 25 to 80° C.) a mucoadhesive film is obtained which can be detached from the support and may be separated by means of punching to yield surface units of 5 cm² each.

### EXAMPLE

| | |
|---|---|
| Na-carboxymethyl cellulose | 52%-wt |
| Hydroxypropyl methyl cellulose | 17%-wt |
| Deoxypeganine hydrochloride | 10%-wt |
| Propanediol | 5%-wt |
| Polyvinyl alcohol | 13%-wt |
| Menthol | 3%-wt |

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. An orally administrable film-shaped medicament containing at least one of the active substance deoxypeganine and a deoxypeganine derivative.

2. The medicament according to claim 1, wherein said medicament contains at least one of a pharmaceutically acceptable salt of deoxypeganine and a pharmaceutically acceptable salt of a derivative of deoxypeganine.

3. The medicament according to claim 1, wherein said medicament is suitable for transmucosal administration of said at least one active substance.

4. The medicament according to claim 1, wherein said medicament has at least one polymer-containing layer which serves as an active substance reservoir and contains said at least one active substance, and wherein said polymer portion is present in an amount between 10 to 90%-wt.

5. The medicament according to claim 1, wherein said medicament has a two-, three- or multilayer structure, wherein at least one layer contains an active substance selected from the group consisting of deoxypeganine, deoxypeganine derivatives and salts of deoxypeganine and deoxvypeanine derivatives.

6. The medicament according to claim 1, wherein the content of said at least one active substance is 0.5 to 40%-wt,

7. The medicament according to claim 1, wherein the overall thickness of said medicament is 0.05 to 3 mm.

8. The medicament according to claim 1, wherein said medicament is mucoadhesive or has at least one mucoadhesive outer surface.

9. The medicament according to claim 1, wherein said medicament is soluble in an aqueous media, and wherein the dissolution takes place between 1 second and 5 minutes.

10. The medicament according to claim 1, wherein said medicament is quickly disintegratable in an aqueous media, and wherein the disintegration takes place within 1 second to 5 minutes.

11. The medicament according to claim 1, wherein said medicament is gelatinizable or swellable in an aqueous media.

12. The medicament according to claim 1, wherein said medicament has a depot effect or releases said at least one active substance with a delay in time over a period of time of up to 8 hours.

13. The medicament according to claim 1, wherein said medicament has at least one rapidly releasing active substance-containing layer and at least one layer with retarded active substance release.

14. The medicament according to claim 1, wherein said medicament additionally contains at least one further pharmaceutically active substance which is not selected from the group consisting of deoxypeganine, deoxypeganine derivatives and salts of deoxypeganine and deoxypeganine derivatives.

15. The film-shaped medicament according to claim 1, wherein said medicament contains at least one auxiliary substance.

16. A use of at least one cholinergic active substance acting on the central nervous system selected from the at least one active substance recited in claim 1 for producing an oral, film-shaped medicament for administering said at least one active substance for treating diseases or symptoms caused by acetylcholine deficiency or where such a deficiency occurs, as well as for treating at least one of diseases where a deficiency of endogenous amine occurs and diseases which can be favourably influenced by inhibition of monoaminoxidase, said use comprising the step of introducing said medicament into the oral cavity at intervals between 1 and 6 hours and wherein the daily dose of said at least one active substance is between 50 and 750 mg.

17. The use according to claim 16, wherein said film-shaped medicament is a medicament according to claim 1.

18. The use according to claim 16, wherein the medicament is used for treating Alzheimer's disease or symptoms caused by Alzheimer's disease.

19. The use according to claim 16, wherein the medicament is used for treating a condition selected from the group of conditions consisting of depressions, schizophrenia and manic disorders.

20. The use according to claim 16, wherein the medicament is used for treating chronic fatigue syndrome or disturbed sleep.

21. The use according to claim 16, wherein the medicament is used for treating abuse of alcohol or for treating abuse of nicotine.

22. The use according to claim 16, wherein the medicament is used for the therapy of abuse of chemical substances, psychotropic substances, or the dependence on such substances.

23. The use according to claim 16, wherein the medicament is used for the prophylactic treatment of poisonings caused by organophosphorous cholinesterase inhibitors.

24. The use according to claim 16, wherein the medicament is used for treating disorders of the central nervous system, which have been caused by the action of psychotropic substances.

25. The medicament according to claim 2, wherein said pharmaceutically acceptable salt of a derivative of deoxypeganine is selected from the group consisting of deoxypeganine hydrochloride and deoxypeganine hydrobromide.

26. The medicament according to claim 3, wherein said medicament is suitable for buccal, administration of said at least one active substance.

27. The medicament according to claim 4, wherein said polymer portion is present in an amount between 20 to 70%-wt.

28. The medicament according to claim 27, wherein said polymer portion is present in an amount between 20 to 60%-wt.

29. The medicament according to claim 6, wherein the content of said at least one active substance is 5 to 30%-wt

30. The medicament according to claim 7, wherein the overall thickness of said medicament is 0.1 to 1 mm.

31. The medicament according to claim 30, wherein the overall thickness of said medicament is 0.1 to 0.5 mm.

32. The medicament according to claim 9, wherein said aqueous media is saliva.

33. The medicament according to claim 9, wherein said dissolution takes place between 3 and 30 seconds.

34. The medicament according to claim 10, wherein said aqueous media is saliva.

35. The medicament according to claim 10, wherein said disintegration takes place within 3 to 30 seconds.

36. The medicament according to claim 11, wherein said aqueous media is saliva.

37. The medicament according to claim 12, wherein said delay in time is a period up to 24 hours.

38. The use according to claim 24, wherein the medicament is used for treating disorders of impaired memory.
